(19) United States
(12) Patent Application Publication
(10) Pub. No.: US 2008/0026050 A1
(43) Pub. Date: Jan. 31, 2008

(54) SOLID DOSE FORMULATIONS OF A THROMBIN RECEPTOR ANTAGONIST

(76) Inventors: Rajan Gupta, Bridgewater, NJ (US); Surendra A. Sangekar, Union, NJ (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(21) Appl. No.: 11/771,571

(22) Filed: Jun. 29, 2007

Related U.S. Application Data
(60) Provisional application No. 60/817,820, filed on Jun. 30, 2006.

(51) Int. Cl. A61K 9/48; A61K 31/44; A61P 9/00; A61K 31/535

(52) U.S. Cl. 424/451; 514/235.2; 514/337

(57) ABSTRACT

Capsule formulations of a thrombin receptor antagonist for oral administration are disclosed. In some embodiments, the thrombin receptor antagonist is COMPOUND 1: 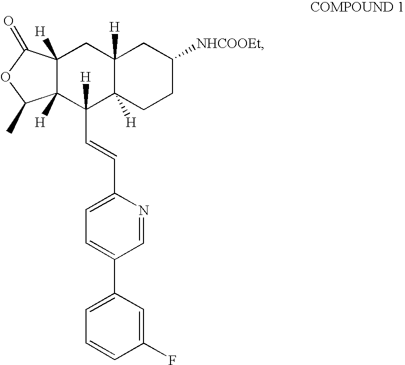 or a pharmaceutically acceptable isomer, salt, or solvate thereof. The formulations include at least one excipient, such as a diluent, disintegrant and/or lubricant. Also disclosed are methods of treating acute coronary syndrome and peripheral arterial disease, and of effecting secondary prevention, by orally administering such capsule formulations.

### CROSS-REFERENCE TO RELATED APPLICATIONS

This application claims the benefit of U.S. provisional application No. 60/817,820, filed on Jun. 30, 2006. Filed contemporaneously with the present application is another nonprovisional application entitled “IMMEDIATE-RELEASE TABLET FORMULATIONS OF A THROMBIN RECEPTOR ANTAGONIST,” attorney docket no. PD06499US, directed to alternate formulations of the same drug substances found in this application.

### FIELD OF THE INVENTION

The invention relates to capsule formulations for delivery of a thrombin receptor antagonist.

### BACKGROUND

Thrombin is known to have a variety of activities in different cell types and thrombin receptors are known to be present in such cell types as human platelets, vascular smooth muscle cells, endothelial cells and fibroblasts. It is therefore possible that thrombin receptor antagonists, also known as protease activated receptor (PAR) antagonists will be useful in the treatment of thrombotic, inflammatory, atherosclerotic and fibroproliferative disorders, as well as other disorders in which thrombin and its receptor play a pathological role.

U.S. application Ser. No. 10/412,982 discloses a specific thrombin receptor antagonist compound identified as Example 2, herein identified as COMPOUND 1. COMPOUND 1 has the following structure. 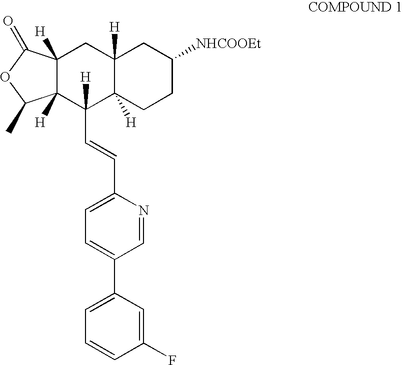
COMPOUND 1 exhibits good thrombin receptor antagonist activity (potency) and selectivity, and is currently in development by Schering Corp. Co-pending U.S. patent application Ser. No. 10/705,282, herein incorporated by reference, discloses a variety of indications and combination formulations for thrombin receptor antagonists including COMPOUND 1. A crystalline form of the bisulfate salt of COMPOUND 1 is disclosed in U.S. Pat. No. 7,235,567.

The use of a small subset of thrombin receptor antagonists to treat a variety of conditions and diseases is disclosed in U.S. publication no. 04/0192753. The prevention of complications associated with cardiopulmonary bypass surgery by administration of a thrombin receptor antagonist is taught in U.S. application Ser. No. 11/613,450. Substituted thrombin receptor antagonists are disclosed in U.S. Pat. Nos. 6,063,847; 6,326,380; and 6,645,987 and U.S. publication nos. 03/0203927; 04/0216437A1; 04/0152736; and 03/0216437. All of the herein cited references are incorporated in their entirety.

It would be beneficial to provide solid dose formulations of acceptable pharmaceutical characteristics for COMPOUND 1. The invention seeks to provide these and other benefits, which will become apparent as the description progresses.

### SUMMARY OF THE INVENTION

In some embodiments, the present invention is directed to a pharmaceutical formulation for oral administration comprising a therapeutically effective amount of a thrombin receptor antagonist, at least one excipient, and a capsule.

In some embodiments, the thrombin receptor antagonist is COMPOUND 1: 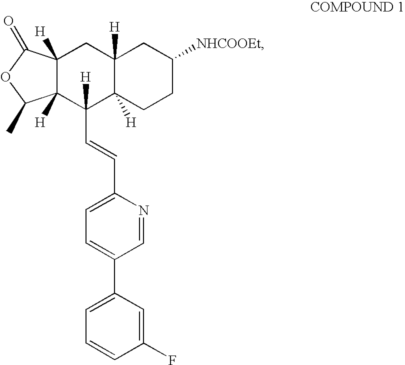
or a pharmaceutically acceptable isomer, salt, or solvate thereof. In some embodiments, the thrombin receptor antagonist is the bisulfate salt of COMPOUND 1.

In some embodiments, the capsule consists of one or more non-gelatin materials. In some embodiments, the one or more non-gelatin materials include hydroxypropyl methylcellulose.

In some embodiments, the therapeutically effective amount is between about 0.25 mg and about 5 mg.

In some embodiments, the therapeutically effective amount is about 2.5 mg.

In some embodiments, the therapeutically effective amount is between about 10 mg and about 50 mg.

In some embodiments, the therapeutically effective amount is about 40 mg.

In some embodiments, the thrombin receptor antagonist is selected from the group consisting of: 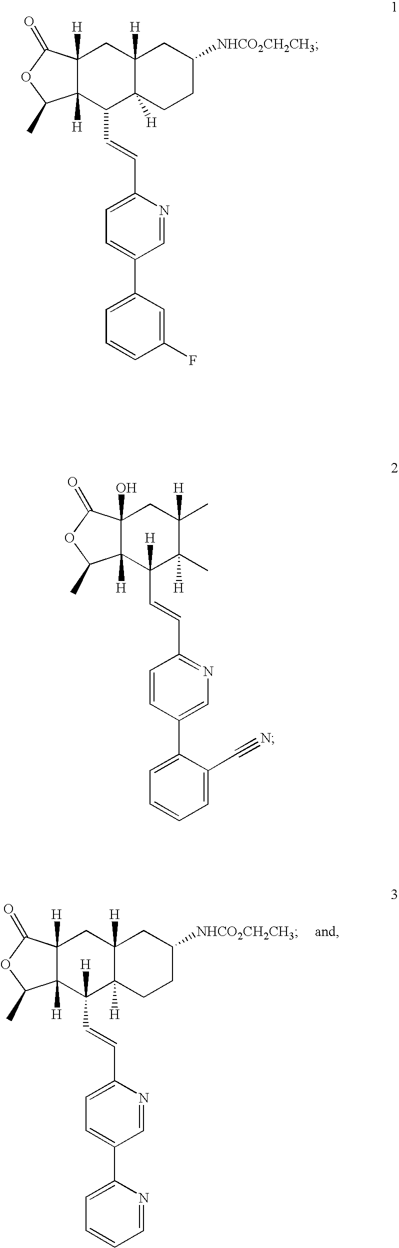 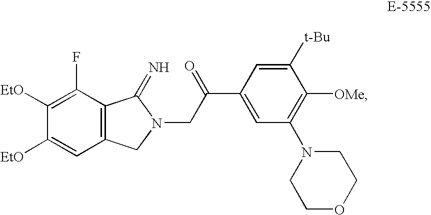
or a pharmaceutically acceptable isomer, salt, or solvate thereof.

In some embodiments, the at least one excipient comprises at least one diluent, at least one disintegrant and at least one lubricant.

In some embodiments, the at least one excipient is a diluent selected from the group consisting of lactose, sucrose, dextrose, mannitol, sorbitol, microcrystalline cellulose, dibasic calcium phosphate, tribasic calcium phosphate, compressible sugar, starch and calcium sulfate. In some embodiments, the diluent is microcrystalline cellulose.

In some embodiments, the at least one excipient is a lubricant selected from the group consisting of magnesium stearate, stearic acid and talc. In some embodiments, the lubricant is magnesium stearate.

In some embodiments, the at least one excipient is a disintegrant selected from the group consisting of crospovidone, microcrystalline cellulose, sodium croscarmelose, starch, sodium carboxymethyl starch, sodium starch glycolate, locust bean, karaya, guar, tragacanth, agar, methylcellulose, sodium carboxymethylcellulose, alginic acid, sodium alginate and bentonite. In some embodiments, the disintegrant is crospovidone.

In some embodiments, the invention is directed to a pharmaceutical formulation comprising about 2.5 mg of COMPOUND 1, between about 25 mg and about 300 mg of a diluent, between about 1 mg and about 50 mg of a disintegrant, and a capsule.

In some embodiments, the invention is directed to a pharmaceutical formulation comprising about 40 mg of COMPOUND 1, between about 25 mg and about 500 mg of a diluent, between about 1 mg and about 75 mg of a disintegrant, and a capsule.

In some embodiments, the invention is directed to a method of treating acute coronary syndrome by orally administering to a patient in need of such treating any of the above pharmaceutical formulations.

In some embodiments, the invention is directed to a method of treating a patient in need of secondary prevention by orally administering to said patient any of the above pharmaceutical formulations.

In some embodiments, the invention is directed to a method of treating peripheral arterial disease by orally administering to a patient in need of such treating any of the above pharmaceutical formulations.

A further understanding of the invention will be had from the following description and claims.

### DESCRIPTION OF THE INVENTION

Schering Corp. is developing a thrombin receptor antagonist (“TRA”) for use in a variety of cardiovascular applications, including treatment of acute coronary syndrome and secondary prevention. The active pharmaceutical ingredient (“API”), COMPOUND 1, has completed phase II clinical trials. Dosing regimens being considered for commercialization include potential loading doses of 10, 20 and 40 mg and maintenance doses of 0.5, 1, 2.5 and 5 mg, in formulations for oral administration. Based on clinical data, it appears that a maintenance dose of between 0.25 and 5 mgs will safely achieve therapeutically effective blood levels of COMPOUND 1 in a patient in the desired time frame. A loading dose of 40 mg and a maintenance dose of 2.5 mg are planned for evaluation in phase III clinical trials. The development of formulations of suitable pharmaceutical characteristics is a necessary step in the commercialization of this thrombin receptor antagonist.

“Acute coronary syndrome” includes any group of clinical symptoms compatible with acute myocardial ischemia. Acute myocardial ischemia is chest pain due to insufficient blood supply to the heart muscle that results from coronary artery disease (also called coronary heart disease). Acute coronary syndrome thus covers the spectrum of clinical conditions ranging from unstable angina to non-Q-wave myocardial infarction and Q-wave myocardial infarction. Symptoms may include chest pain, shortness of breath, nausea, vomiting, diaphoresis (sweating), palpitations, anxiety or a sense of impending doom and a feeling of being acutely ill.

“Secondary prevention” refers to the treatment of patients who have already suffered a significant cardiovascular event, such as a heart attack or stroke, to prevent another future, potentially more serious, perhaps lethal, cardiovascular or cerebrovascular event.

Another cardiovascular condition for which thrombin receptor antagonists may be useful is peripheral arterial disease (“PAD”), also known as peripheral vascular disease (“PVD”), which occurs when cholesterol and scar tissue build up, forming plaque inside the arteries that narrows and clogs the arteries. The clogged arteries cause decreased blood flow to the legs, which can result in pain when walking, and eventually gangrene and amputation.

For the first-in-human, phase I studies, 0.25-, 1.0-, and 5.0-mg capsule formulations of COMPOUND 1 were developed using a simple dry blending process. The prototype formulations were developed based on the results of excipient compatibility studies, dissolution screening studies and content uniformity studies. These prototype formulations, labeled “A,” “B” and “C” are displayed in Table 1.

TABLE 1
Prototype Capsule Formulations

| | | Formulation | | |
|---|---|---|---|---|
| | | Amount (mg) | | |
| Ingredient | Function | A | B | C |
| COMPOUND 1 | TRA | 0.25 | 1.0 | 5.0 |
| (bisulfate) | | | | |
| Microcrystalline | Diluent | 140.75 | 140.0 | 136.0 |
| Cellulose | | | | |
| Crospovidone | Disintegrant | 7.5 | 7.5 | 7.5 |
| Magnesium Stearate | Lubricant | 1.5 | 1.5 | 1.5 |
| Capsule Fill Weight | | 150.0 | 150.0 | 150.0 |
| Capsule, No. 2 Blue | Capsule Shell | | | |
| Opaque Gelatin | | | | |

The prototype formulations were tested for quality attributes at initial and accelerated stability conditions. The formulations exhibited acceptable content uniformity and dissolution at initial and one-month storage at 25° C./60% relative humidity. However, a significant impact on dissolution was observed within a week upon storage at 40° C./75% relative humidity condition. It was hypothesized that this was due to a cross-linking of the gelatin capsule shell, which resulted in a hardened capsule shell. A cross-linking of the capsule shell is possible due to a reaction between gelatin and bisulfate ion. Based on these findings, a long-term storage at 4±2° C. was required for the phase I gelatin-based capsule formulations. However, such conditions are not desirable in a commercial product, and alternatives were sought. Thus, a commercial formulation of COMPOUND 1 bisulfate can be achieved by using non-gelatin based capsules, (e.g., hydroxypropyl methylcellulose (“HPMC”) or carrageenan based capsules).

Non-gelatin capsules greatly reduce the cross-linking that occurs between a capsule and fill material. Cross-linking often results in the formation of a pellicle, or membrane between drug and capsule that can retard dissolution. Vegetable-based, (i.e., non-gelatin) capsules are typically less prone to cross-linking than are gelatin-based capsules, and offer greater flexibility in processing conditions.

The three capsule formulations displayed in Table 1 were developed for, and administered in, phase I clinical trials. Subsequently, phase II trials were conducted and the results of these trials have lead investigators to select doses of 2.5 mg and 40 mg for maintenance and loading doses, respectively, in phase III trials. Thus, formulators used the knowledge gained in the development of the phase I capsule formulations to project capsule formulations that would correspond to the doses to be administered in phase III trials. Based on the on the results of excipient compatibility studies, dissolution screening studies and content uniformity studies that resulted in the above formulations, a 2.5 mg maintenance dose formulation was conceived as displayed in Table 2.

TABLE 2
Concept 2.5 mg Formulation

| | Amount |
|---|---|
| Ingredient | (mg) |
| COMPOUND 1 (bisulfate) | 2.5 |
| Microcrystalline Cellulose | 138.5 |
| Crospovidone | 7.5 |
| Magnesium Stearate | 1.5 |
| Capsule Fill Weight | 150.0 |
| Non-Gelatin capsule, e.g., V-caps (HPMC | |
| capsules) | |

Similarly, 40 mg loading dose formulations were conceived as displayed in Table 3.

TABLE 3
Concept 40 mg Formulations

| | |
|---|---|
| | Amount |
| Ingredient | (mg) |

| | | |
|---|---|---|
| COMPOUND 1 (bisuifate) | 40.0 | 40.0 |
| Microcrystalline Cellulose | 242.0 | 336.0 |
| Crospovidone | 15.0 | 20.0 |
| Magnesium Stearate | 3.0 | 4.0 |
| Capsule Fill Weight | 300.0 | 400.0 |
| Non-gelatin capsule, e.g., V-caps (HPMC | Size 0 | Size 00 |
| capsules) | | |

Although based on the development studies that resulted in the 0.25-, 1.0- and 5.0-mg prototype formulations, the concept formulations of Tables 3 and 4 were not prepared.

The formulations of the present invention comprise solid forms of a thrombin receptor antagonist and at least one excipient selected from a variety of pharmaceutically acceptable excipients contained in a capsule. As illustrated in the exemplified formulations displayed in Tables 1-3, the formulation may contain such excipients as a diluent, a disintegrant and a lubricant. In some embodiments, maintenance dose formulations of COMPOUND 1 comprise about 2.5 mg of COMPOUND 1, between about 25 mg and about 300 mg of a diluent, and between about 1 mg and about 50 mg of a disintegrant. In some embodiments, loading dose formulations of COMPOUND 1 comprise about 40 mg of COMPOUND 1, between about 25 mg and about 500 mg of a diluent, and between about 1 mg and about 75 mg of a disintegrant.

Preferred diluents include sugars, such as lactose, sucrose, dextrose, mannitol, and sorbitol, microcrystalline cellulose, tribasic calcium phosphate, dibasic calcium phosphate, compressible sugar, starch and calcium sulfate.

As used herein for solid oral dosage forms of the present invention, the term “disintegrant” refers to a substance added to the dosage form to help it break apart (disintegrate) and release the medicinal agent(s). Suitable disintegrants include crospovidone, microcrystalline cellulose, sodium croscarmelose, starch, sodium carboxymethyl starch, sodium starch glycolate, locust bean, karaya, guar, tragacanth, agar, methylcellulose, sodium carboxymethylcellulose, alginic acid, sodium alginate and bentonite.

Preferred lubricants may include magnesium stearate, stearic acid and talc.

As used herein, the term “formulation” is intended to encompass a product comprising the specified ingredients in the specified amounts, as well as any product which results, directly or indirectly, from combination of the specified ingredients in the specified amounts.

“Effective amount” or “therapeutically effective amount” is meant to describe an amount of the thrombin receptor antagonist that produces the desired therapeutic, ameliorative or preventative effect. Therapeutically effective amounts of COMPOUND 1 are believed to be between about 0.25 mg and about 50 mg, preferably between about 0.5 and 5.0 for the maintenance dose and between about 10 and about 50 mg for the loading dose. More preferred maintenance and loading doses are about 2.5 and about 40 mg, respectively.

The present invention encompasses solid formulations of any thrombin receptor antagonist. A variety of compounds have been demonstrated as displaying activity as thrombin receptor antagonists, many being himbacine analogs. As disclosed in U.S. publication no. 04/0152736, a subset of particularly preferred compounds of Formula I is as follows: 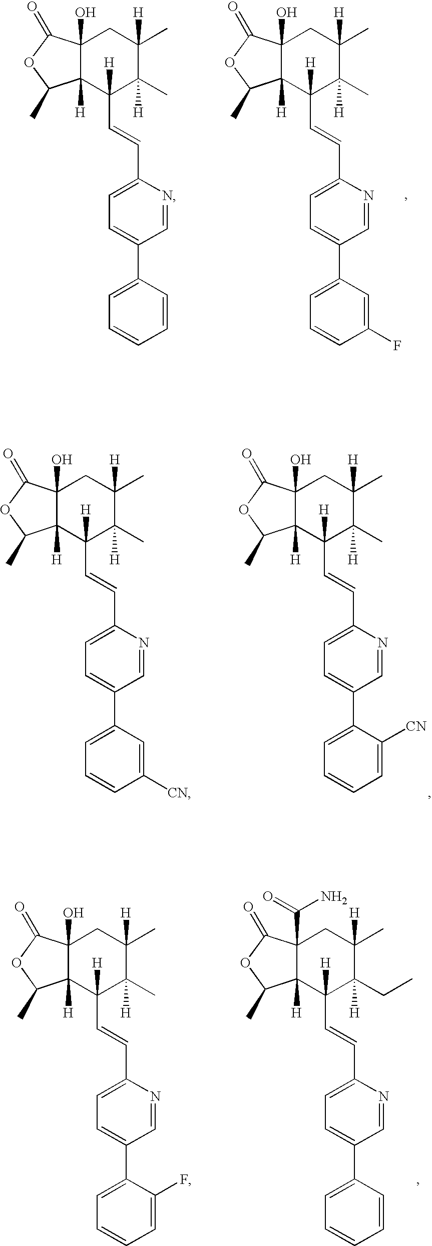 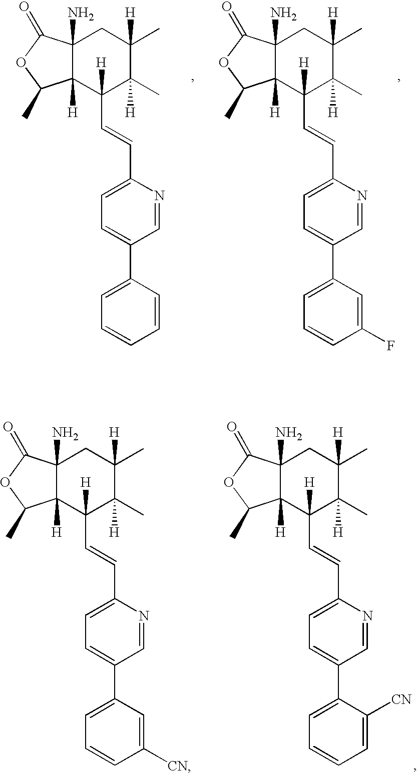
and the pharmaceutically acceptable isomers, salts, solvates and polymorphs thereof. U.S. publication no. 03/0216437 discloses a subset of thrombin receptor antagonists of Formula II which are both particularly active and selective. These compounds are as follows: 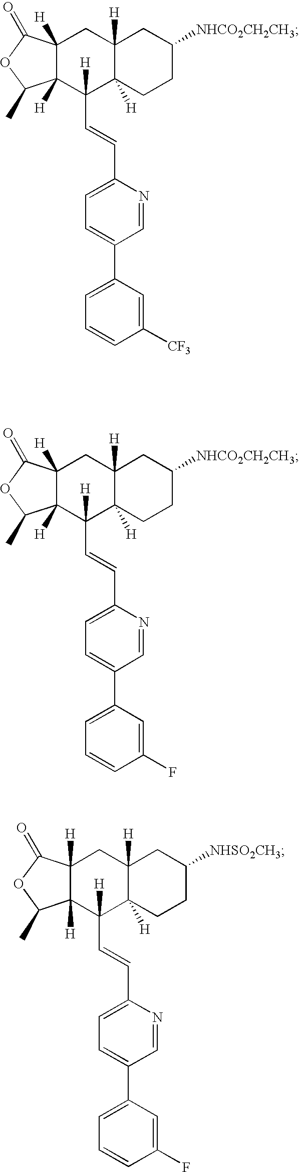 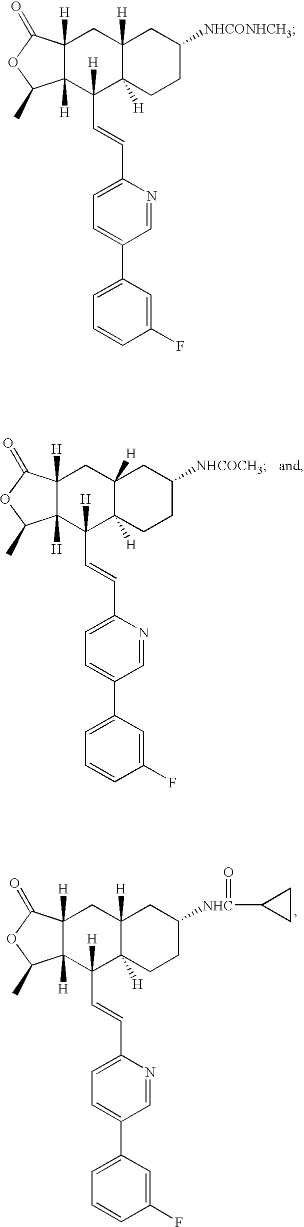
and the pharmaceutically acceptable isomers, salts, solvates and polymorphs thereof.

The following compounds are particularly favored based on their pharmacokinetics and pharmacodynamic characteristics: 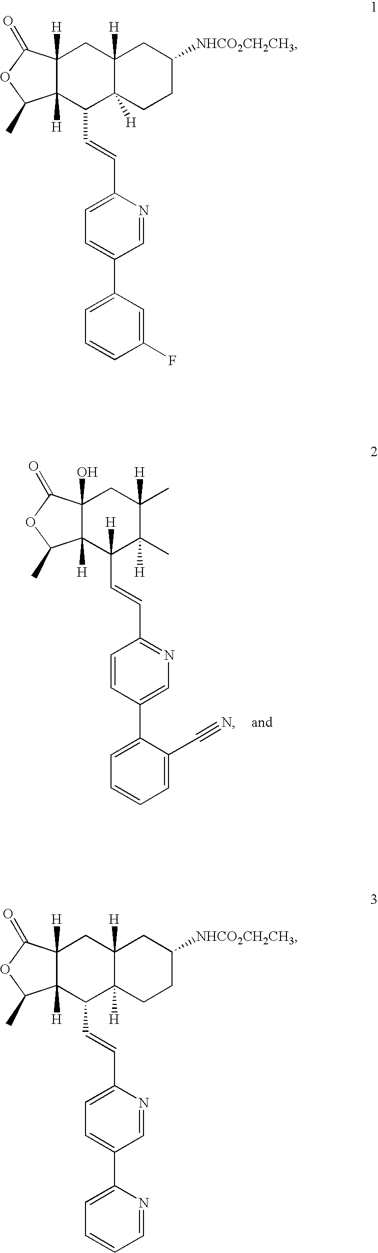
and the pharmaceutically acceptable isomers, salts, solvates and polymorphs thereof. The bisulfate salt of COMPOUND 1 is currently in development as a thrombin receptor antagonist by Schering Corp. Its synthesis is disclosed in U.S. publication no. 0310216437, published Nov. 20, 2003, which publication also discloses Compound 3. Compound 2 is disclosed in U.S. Pat. No. 6,645,987.

Other compounds for use in the combinations of the present invention are disclosed in any of U.S. Pat. Nos. 6,063,847 and 6,326,380, U.S. Patent Publications 03/0203927, 03/0216437, 04/0192753 and 04/0176418, all of which are incorporated by reference in their entirety. Combinations that include one or more other agents that display activity as thrombin receptor antagonists are also within the scope of the present invention, including E5555 currently in development by Eisai:

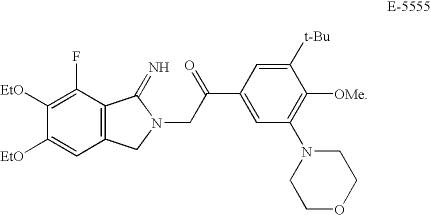

It will be understood that unless otherwise specified, the term “thrombin receptor antagonist:” and any compounds identified as such, including COMPOUND 1, encompass any chemically stable and pharmaceutically acceptable free base, salt, isomer or solvate form thereof. The term “salt(s)”, as employed herein, denotes acidic salts formed with inorganic and/or organic acids. Pharmaceutically acceptable (i.e., non-toxic, physiologically acceptable) salts are preferred, although other salts are also useful. Salts of the compound of the above active agents may be formed, for example, by reacting the above active agents with an equivalent amount of acid or base in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates) and the like. Additionally, acids which are generally considered suitable for the formation of pharmaceutically useful salts from basic pharmaceutical compounds are discussed, for example, by S. Berge et al, Journal of Pharmaceutical Sciences (1977) (66)1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

All such acid salts are intended to be pharmaceutically acceptable salts within the scope of the invention and all acid and base salts are considered equivalent to the free forms of the corresponding compounds for purposes of the invention.

Thrombin receptor antagonists for use in formulations of the present invention, and salts and solvates thereof, may exist in their tautomeric form (for example, as an amide or imino ether). All such tautomeric forms are contemplated herein as part of the present invention.

All isomers, including diastereomers and rotational isomers are contemplated as being part of this invention. The invention includes (+)- and (−)-isomers in both pure form and in admixture, including racemic mixtures. All stereoisomers (for example, geometric isomers, optical isomers and the like) of the present compounds (including those of the salts and solvates of the compounds), such as those which may exist due to asymmetric carbons on various substituents, including enantiomeric forms (which may exist even in the absence of asymmetric carbons), rotameric forms, atropisomers, and diastereomeric forms, are contemplated within the scope of this invention. Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt” and “solvate” and the like, is intended to equally apply to the salt, solvate and prodrug of enantiomers, stereoisomers, rotamers, tautomers, racemates or prodrugs of the inventive compounds.

The term “solvate” will be understood to encompass hydrates.

Manufacturing of these formulations involves dry blending of active with other ingredients, preferably by geometric dilution technique. Where the formulation is in the form of a capsule, these steps are followed by encapsulation of the dry blend in suitable size gelatin or non-gelatin, e.g., HPMC, capsule shells. The capsules are then packaged in high density polyethylene bottles with induction seal and child-resistant caps, and are stored under refrigeration.

By way of example, a capsule formulation containing 0.25 mg of COMPOUND 1 can be prepared in batch as follows.

TABLE 4

| | Amount | Amount (g/40,000 |
|---|---|---|
| Ingredient | (mg/capsule) | capsules) |
| (1) Active | 0.25 | 10 |
| (2) Microcrystalline Cellulose PH101 | 140.75 | 5630 |
| (3) Crospovidone | 7.5 | 300 |
| (4) Magnesium Stearate (non- | 1.5 | 60 |
| bovine) | | |
| Capsule Fill Weight | 150.0 | 40,000 capsules |

1. Weigh all the ingredients (Items 1-4) to approximately 5-10% excess of the required quantities.
2. Pass all the ingredients in Step 1 through a 40 mesh screen.
3. Weigh the requisite quantities of the screened ingredients as per the batch formula.
4. Prepare a premix of the active with approximately˜200 g of microcrystalline cellulose (Item No. 2) on a screen pan with spatula (Pan size: 12″).
5. Pass the premix in Step 4 through a 40 mesh screen three times.
6. Charge the screened mixture from Step 5 in a 5-L Bohle Blender bin. Blend for 10 minutes at 30±2 rpm.
7. Add approximately 900 g of microcrystalline cellulose in Step 3 to the bin in Step 6.
8. Blend for 10 minutes in the Bohle blender at 30±2 rpm.
9. Pass the blend in Step 8 through a 40 mesh screen.
10. Transfer the blend in Step 9 to a 20-L Bohle Blender bin.
11. Rinse the 5-L bin in Step 8 with a portion of the remaining microcrystalline cellulose (500 g) in Step 3 at least 3 times. Charge the material to the 20-L bin in Step 10.
12. Charge the crospovidone in Step 3 and the remaining microcrystalline cellulose to the bin in Step 11. Blend for 10 minutes at 50±2 rpm.
13. Pass the blend in Step 12 through a 40 mesh screen.
14. Return the screened blend to the 20-L bin. Blend for 10 minutes at 30±2 rpm.
15. Remove an amount of material from the blender bin in Step 14 that is approximately equivalent in volume to the magnesium stearate (˜60 g).
16. Prepare a premix of the material in Step 15 with the screened magnesium stearate in Step 3.
17. Charge the premix in Step 16 to the 20-L bin in Step 14. Blend for 5 minutes at 30±2 rpm.
18. Transfer the contents of the blender into appropriate bulk storage containers (e.g., cardboard box double-line with polyethylene bags and desiccant between the bags).
19. Fill the mix into a suitable size colored capsules using a Bosch 400 capsule filling machine (preferred dosing disk: 12 mm).
20. Pass the capsules through a suitable capsule polishing equipment (e.g., Nutting capsule polisher).
21. Inspect the polished capsules for physical defects, e.g., dents, cracks and dullness.
22. Collect the polished capsules in labeled, double polyethylene bag-lined containers with a desiccant bag between the bags (see below).

Capsules should be stored for 72 hours under controlled room temperature to complete the packaging step. Refrigerate the batch if time between the end of capsule filling and the end of packaging cannot be completed within 72 hours.

By way of further example, capsules may be filled according to the following procedure:
1. Fill the mix into a suitable size colored capsule using a Bosch 400 capsule filling machine (preferred dosing disk: 12 mm).
2. Pass the capsules through a suitable capsule polishing equipment (e.g., Nutting capsule polisher).
3. Inspect the polished capsules for physical defects, e.g., dents, cracks and dullness.
4. Collect the polished capsules in labeled, double polyethylene bag-lined containers with a desiccant bag between the bags.

Bulk storage of capsules (Step 18) should be continued for 72 hours under controlled room temperature to complete the packaging step. Refrigerate the batch if time between the end of capsule filling and the end of packaging cannot be completed within 72 hours.

While the present invention has been described in conjunction with the specific embodiments set forth above, many alternatives, modifications and variations thereof will be apparent to those of ordinary skill in the art. All such alternatives, modifications, and variations are intended to fall within the spirit and scope of the present invention.

What is claimed is:

1. A pharmaceutical formulation for oral administration comprising a therapeutically effective amount of a thrombin receptor antagonist, at least one excipient, and a capsule.

2. The formulation according to claim 1, wherein said thrombin receptor antagonist is COMPOUND 1: 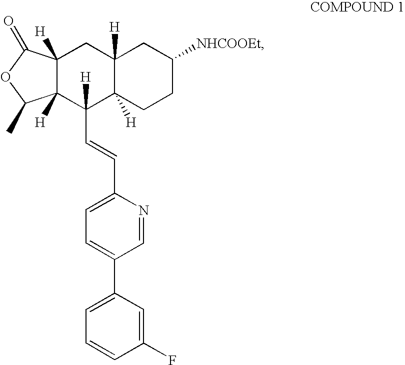
or a pharmaceutically acceptable isomer, salt, or solvate thereof.

3. The formulation according to claim 1, wherein said thrombin receptor antagonist is the bisulfate salt of COMPOUND 1:

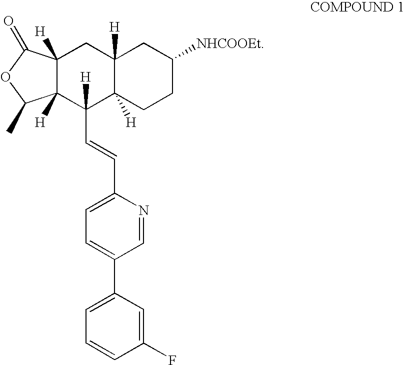

4. The formulation according to claim 3, wherein said capsule consists of one or more non-gelatin materials.

5. The formulation according to claim 4, wherein said one or more non-gelatin materials include hydroxypropyl methylcellulose.

6. The formulation according to any of claims 2 and 3, wherein said therapeutically effective amount is between about 0.25 mg and about 5 mg.

7. The formulation according to any of claims 2 and 3, wherein said therapeutically effective amount is about 2.5 mg.

8. The formulation according to any of claims 2 and 3, wherein said therapeutically effective amount is between about 10 mg and about 50 mg.

9. The formulation according to any of claims 2 and 3, wherein said therapeutically effective amount is about 40 mg.

10. The formulation according to claim 1, wherein said thrombin receptor antagonist is selected from the group consisting of: 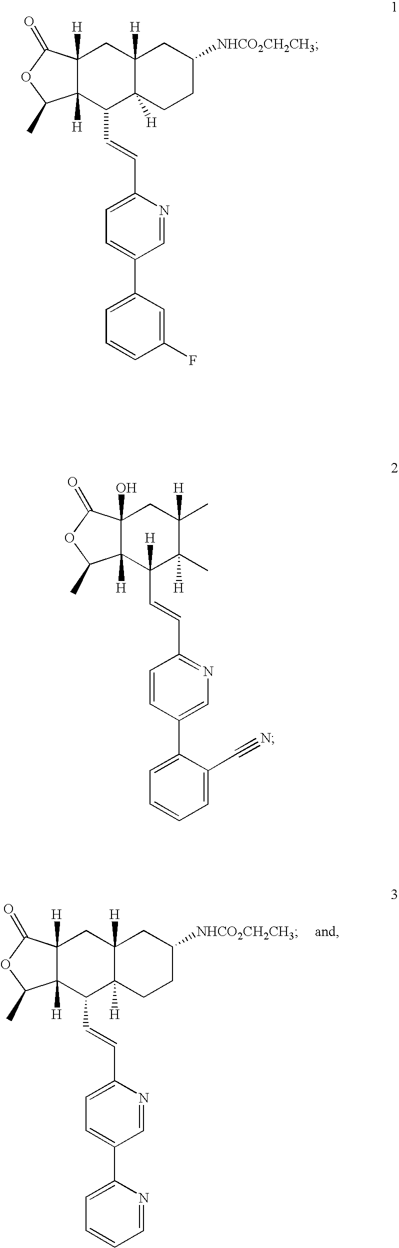 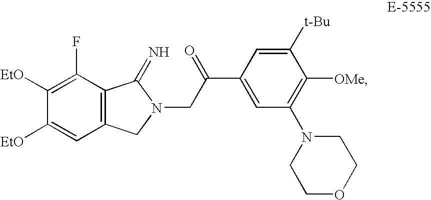
or a pharmaceutically acceptable isomer, salt, or solvate thereof.

11. The formulation according to any of claims 1-3, wherein said at least one excipient comprises at least one diluent, at least one disintegrant and at least one lubricant.

12. The pharmaceutical formulation according to claim 1, wherein said at least one excipient is a diluent selected from the group consisting of lactose, sucrose, dextrose, mannitol, sorbitol, microcrystalline cellulose, dibasic calcium phosphate, tribasic calcium phosphate, compressible sugar, starch and calcium sulfate.

13. The pharmaceutical formulation according to claim 12, wherein said diluent is microcrystalline cellulose.

14. The pharmaceutical formulation according to claim 1, wherein said at least one excipient is a lubricant selected from the group consisting of magnesium stearate, stearic acid and talc.

15. The pharmaceutical formulation according to claim 14, wherein said lubricant is magnesium stearate.

16. The pharmaceutical formulation according to claim 1, wherein said at least one excipient is a disintegrant selected from the group consisting of crospovidone, microcrystalline cellulose, sodium croscarmelose, starch, sodium carboxymethyl starch, sodium starch glycolate, locust bean, karaya, guar, tragacanth, agar, methylcellulose, sodium carboxymethylcellulose, alginic acid, sodium alginate and bentonite.

17. The pharmaceutical formulation according to claim 16, wherein said disintegrant is crospovidone.

18. A pharmaceutical formulation comprising about 2.5 mg of COMPOUND 1, between about 25 mg and about 300 mg of a diluent, between about 1 mg and about 50 mg of a disintegrant, and a capsule.

19. A pharmaceutical formulation comprising about 40 mg of COMPOUND 1, between about 25 mg and about 500 mg of a diluent, between about 1 mg and about 75 mg of a disintegrant, and a capsule.

20. A method of treating acute coronary syndrome by orally administering to a patient in need of such treating the pharmaceutical formulation according to any of claims 1, 2, 3, and 10.

21. A method of treating a patient in need of secondary prevention by orally administering to said patient the pharmaceutical formulation according to any of claims 1, 2, 3 and 10.

22. A method of treating peripheral arterial disease by orally administering to a patient in need of such treating the pharmaceutical formulation according to any of claims 1, 2, 3 and 10.
